(19) United States
(12) Patent Application Publication
(10) Pub. No.: US 2009/0131422 A1
(43) Pub. Date: May 21, 2009

(54) Salts of an IKK inhibitor

(75) Inventors: Frederick A. Hicks, Somerville, MA (US); Martin Ian Cooper, Foxton (GB); Adrian St Clair Brown, Ely (GB)

Correspondence Address: MILLENNIUM PHARMACEUTICALS, INC., 40 Landsdowne Street, CAMBRIDGE, MA, 02139, US

(73) Assignee: Millennium Pharmaceuticals, Inc., Cambridge, MA (US)

(21) Appl. No.: 12/288,595

(22) Filed: Oct. 22, 2008

Related U.S. Application Data
(60) Provisional application No. 61/000,051, filed on Oct. 23, 2007.

(51) Int. Cl. A61K 31/5377; C07D 413/14; A61P 29/00

(52) U.S. Cl. 514/232.5; 544/80

(57) ABSTRACT

The present invention is directed to the Tartrate, Mono-Hydrochloride, Malonate and p-Toluenesulfonate Salts of the compound of formula (I),

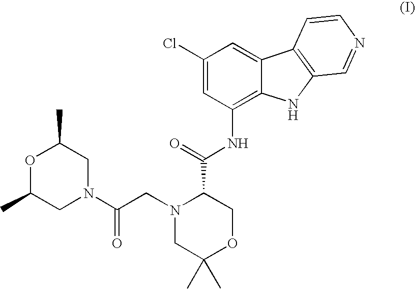

or solvates thereof, or crystalline forms thereof; to a pharmaceutical composition comprising a pharmaceutically effective amount of the Salts, including crystalline forms thereof, and a pharmaceutically acceptable carrier; and to the use of the Salts, including crystalline forms thereof, for treating a patient suffering from, or subject to, a pathological condition capable of being ameliorated by inhibiting IKK-2, and methods related thereto.

### PRIORITY CLAIM

This application claims priority from U.S. Provisional Patent Application No. 61/000,051, filed Oct. 23, 2007, which is hereby incorporated by reference in its entirety.

### FIELD OF THE INVENTION

The present invention is directed to compounds of structural formulas (IIa), (IIb), (IIc) and (IId):

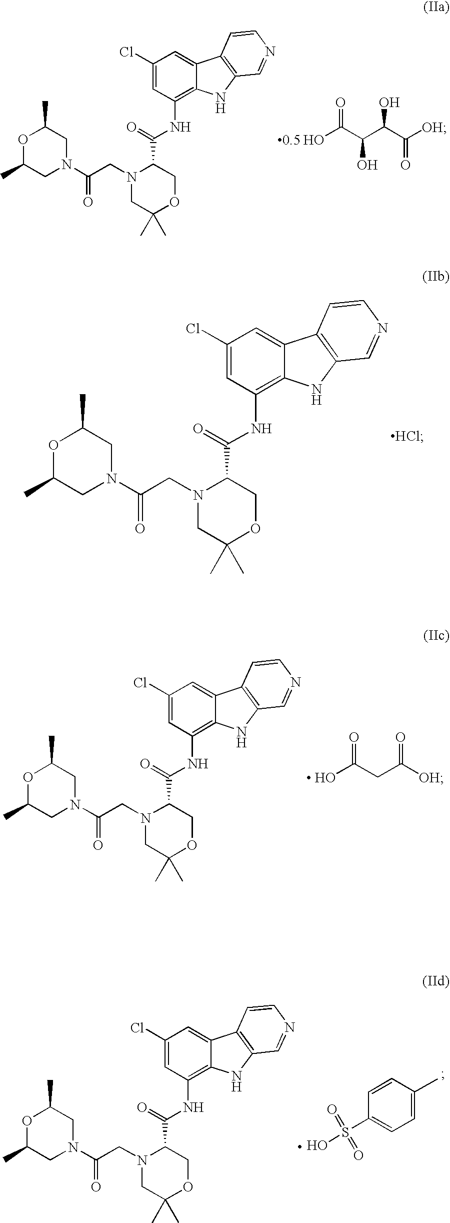

or solvates thereof.

The invention is also directed to the pharmaceutical use of the compounds as IκB inhibitors, crystalline forms thereof, and pharmaceutical compositions comprising the compounds of the invention.

As an inhibitor of IκB kinase, the compounds of the invention function via the selective inhibition of IKK, particularly an IKK-2 inhibitor. Such an inhibitor is particularly useful for treating a patient suffering from or subject to IKK-2 mediated pathological diseases or conditions, e.g., joint inflammation (e.g., rheumatoid arthritis (RA), rheumatoid spondylitis, gouty arthritis, traumatic arthritis, rubella arthritis, psoriatic arthritis, osteoarthritis, and other arthritic conditions), acute synovitis, tuberculosis, atherosclerosis, muscle degeneration, cachexia, Reiter's syndrome, endotoxaemia, sepsis, septic shock, endotoxic shock, gram negative sepsis, gout, toxic shock syndrome, pulmonary inflammatory diseases (e.g., asthma, acute respiratory distress syndrome, chronic obstructive pulmonary disease, silicosis, pulmonary sarcoidosis, and the like), bone resorption diseases, reperfusion injuries, carcinoses, leukemia, sarcomas, lymph node tumors, skin carcinoses, apoptosis, graft versus host reaction, graft versus host disease (GVHD), allograft rejection, leprosy, viral infections (e.g., HIV, cytomegalovirus (CMV), influenza, adenovirus, the Herpes group of viruses, and the like), parasitic infections (e.g., malaria, such as cerebral malaria), yeast and fungal infections (e.g., fungal meningitis), fever and myalgias due to infection, acquired immune deficiency syndrome (AIDS), AIDS related complex (ARC), cachexia secondary to infection or malignancy, cachexia secondary to AIDS or cancer, keloid and scar tissue formation, pyresis, diabetes, inflammatory bowel diseases (IBD) (e.g., Crohn's disease and ulcerative colitis), multiple sclerosis (MS), ischemic brain injury, e.g. cerebral infarction (stroke), head trauma, psoriasis, Alzheimer's disease, carcinomatous disorders (potentiation of cytotoxic therapies), cardiac infarct, chronic obstructive pulmonary disease (COPD), COPD exacerbations, acute respiratory distress syndrome (ARDS), and cancer (e.g., lymphoma, such as diffuse large B-cell, primary mediastinal B-cell, and mantle cell; multiple myeloma; osteolytic bone metastasis; head and neck squamous cell cancer; prostate cancer; pancreatic cancer and non-small cell lung cancer), to name a few, that could be ameliorated by the targeted administration of the inhibitor.

### Reported Developments

NF-κB is a heterodimeric transcription factor that regulates the expression of multiple inflammatory genes. NF-κB has been implicated in many pathophysiologic processes including angiogenesis (Koch et al., Nature 1995, 376, 517-519), atherosclerosis (Brand et al., J Clin Inv. 1996, 97, 1715-1722), endotoxic shock and sepsis (Bohrer et al., J. Clin. Inv. 1997, 100, 972-985), inflammatory bowel disease (Panes et al., Am J. Physiol. 1995, 269, H1955-H1964), ischemia/reperfusion injury (Zwacka et al., Nature Medicine 1998, 4, 698-704), and allergic lung inflammation (Gosset et al., Int Arch Allergy Immunol. 1995, 106, 69-77). Thus the inhibition of NF-κB by targeting regulatory proteins in the NF-κB activation pathway represents an attractive strategy for generating anti-inflammatory therapeutics due to NF-κB's central role in inflammatory conditions.

The IκB kinases (IKKs) are key regulatory signaling molecules that coordinate the activation of NF-κB. Many immune and inflammatory mediators including TNFα, lipopolysaccharide (LPS), IL-1β, CD3/CD28 (antigen presentation), CD40L, FasL, viral infection, and oxidative stress have been shown to lead to NF-κB activation. Although the receptor complexes that transduce these diverse stimuli appear very different in their protein components, it is understood that each of these stimulation events leads to activation of the IKKs and NF-κB.

The IKK complex appears to be the central integrator of diverse inflammatory signals leading to the phosphorylation of IκB. Cell and animal experiments indicate that IKK-2 is a central regulator of the pro-inflammatory role of NF-κB, wherein the IKK-2 is activated in response to immune and inflammatory stimuli and signaling pathways. Many of those immune and inflammatory mediators, including IL-1β, LPS, TNFα, CD3/CD28 (antigen presentation), CD40L, FasL, viral infection, and oxidative stress, play an important role in respiratory diseases. Furthermore, the ubiquitous expression of NF-κB, along with its response to multiple stimuli means that almost all cell types present in the lung are potential targets for anti-NF-κB/IKK-2 therapy. This includes alveolar epithelium, mast cells, fibroblasts, vascular endothelium, and infiltrating leukocytes, including neutrophils, macrophages, lympophocytes, eosinophils and basophils.

Inhibitors of IKK-2 are believed to display broad anti-inflammatory activity by inhibiting the expression of genes such as cyclooxygenase-2 and 12-lipoxygenase (synthesis of inflammatory mediators), TAP-1 peptide transporter (antigen processing), MHC class I H-2K and class II invariant chains (antigen presentation), E-selectin and vascular cell adhesion molecule (leukocyte recruitment), interleukins-1,2,6,8 (cytokines), RANTES, eotaxin, GM-CSF (chemokines), and superoxide dismutase and NADPH quinone oxidoreductase (reactive oxygen species).

NF-κB is activated beyond its normal extent in diseases such as rheumatoid arthritis, osteoarthritis, asthma, chronic obstructive pulmonary disease (COPD), rhinitis, multiple sclerosis, cardiac infarction, Alzheimer's diseases, diabetes Type II, inflammatory bowel disease or atherosclerosis.

The inhibition of NF-κB is also described as being useful for treating hypoproliferative diseases, e.g., solid tumor and leukemias, on its own or in addition to cytostatic therapy. Inhibition of the NF-κB-activating signal chain at various points or by interfering directly with the transcription of the gene by glucocorticoids, salicylates or gold salts, has been shown as being useful for treating rheumatism.

Patent applications WO04/092167, US2004-0235839, WO05/111037 and US2005-0239781 disclose beta-carboline compounds that exhibit an inhibitory effect on IKK. These applications additionally disclose methods for the preparation of these compounds, pharmaceutical compositions containing these compounds, and methods for the prophylaxis and therapy of diseases, disorders, or conditions associated with an increased activity of IκB kinase, including but not limited to rheumatoid arthritis and multiple sclerosis.

(S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide (I) is also specifically disclosed:

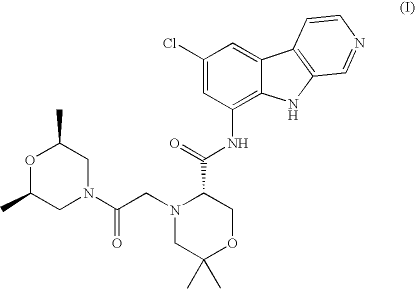

The structure and synthesis of the free-base amorphous form of this compound is provided in the working examples in WO04/092167, US2004-0235839, WO05/111037 and US2005-0239781, and only a general discussion of a wide variety of salts is disclosed. These applications do not disclose specific salts or crystalline forms of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide.

The large-scale manufacturing of a pharmaceutical composition poses many challenges to the chemist and chemical engineer. While many of these challenges relate to the handling of large quantities of reagents and control of large-scale reactions, the handling of the final product poses special challenges linked to the nature of the final active product itself. Not only must the product be prepared in high yield, be stable, and capable of ready isolation, the product must possess properties that are suitable for the types of pharmaceutical preparations in which they are likely to be ultimately used. The stability of the active ingredient of the pharmaceutical preparation must be considered during each step of the manufacturing process, including the synthesis, isolation, bulk storage, pharmaceutical formulation and long-term formulation. Each of these steps may be impacted by various environmental conditions of temperature and humidity.

The pharmaceutically active substance used to prepare the pharmaceutical compositions should be as pure as possible and its stability on long-term storage must be guaranteed under various environmental conditions. These properties are absolutely essential to prevent the appearance of unintended degradation products in pharmaceutical compositions, which degradation products may be potentially toxic or result simply in reducing the potency of the composition.

A primary concern for the manufacture of large-scale pharmaceutical compounds is that the active substance should have a stable crystalline morphology to ensure consistent processing parameters and pharmaceutical quality. If an unstable crystalline form is used, crystal morphology may change during manufacture and/or storage resulting in quality control problems, and formulation irregularities. Such a change may affect the reproducibility of the manufacturing process and thus lead to final formulations which do not meet the high quality and stringent requirements imposed on formulations of pharmaceutical compositions. In this regard, it should be generally borne in mind that any change to the solid state of a pharmaceutical composition which can improve its physical and chemical stability gives a significant advantage over less stable forms of the same drug.

When a compound crystallizes from a solution or slurry, it may crystallize with different spatial lattice arrangements, a property referred to as “polymorphism.” Each of the crystal forms is a “polymorph.” While polymorphs of a given substance have the same chemical composition, they may differ from each other with respect to one or more physical properties, such as solubility and dissociation, true density, melting point, crystal shape, compaction behavior, flow properties, and/or solid state stability.

As described generally above, the polymorphic behavior of drugs can be of great importance in pharmacy and pharmacology. The differences in physical properties exhibited by polymorphs affect practical parameters such as storage stability, compressibility and density (important in formulation and product manufacturing), and dissolution rates (an important factor in determining bio-availability). Differences in stability can result from changes in chemical reactivity (e.g., differential oxidation, such that a dosage form discolors more rapidly when it is one polymorph than when it is another polymorph) or mechanical changes (e.g., tablets crumble on storage as a kinetically favored polymorph converts to thermodynamically more stable polymorph) or both (e.g., tablets of one polymorph are more susceptible to breakdown at high humidity). In addition, the physical properties of the crystal may be important in processing: for example, one polymorph might be more likely to form solvates that cause the solid form to aggregate and increase the difficulty of solid handling, or might be difficult to filter and wash free of impurities (i.e., particle shape and size distribution might be different between one polymorph relative to other).

While drug formulations having improved chemical and physical properties are desired, there is no predictable means for preparing new drug forms (e.g., polymorphs) of existing molecules for such formulations. These new forms would provide consistency in physical properties over a range of environments common to manufacturing and composition usage. More particularly, there is a need for an inhibitor of IκB kinase that operates through the selective inhibition of IKK, particularly an IKK-2 inhibitor. Such an inhibitor should have utility in treating a patient suffering from or subject to IKK-2 mediated pathological (diseases) conditions, e.g., rheumatoid arthritis or multiple sclerosis, as well as having properties suitable for large-scale manufacturing and formulation.

In the instant case, no art discloses or teaches salts of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide, or crystalline forms thereof. More particularly, no art discloses or teaches salts of (S)—N-(6-chloro-9H-pyrido[3,4-b]indol-8-yl)-4-(2-((2S,6R)-2,6-dimethylmorpholino)-2-oxoethyl)-6,6-dimethylnorpholine-3-carboxamide, or crystalline forms thereof, that are particularly useful for large-scale manufacturing, pharmaceutical formulation, and storage.

### SUMMARY OF THE INVENTION

The present invention is directed to salts of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide, or crystalline forms thereof. Those forms also have properties that are useful for large-scale manufacturing, pharmaceutical formulation, and storage. The present invention also provides pharmaceutical compositions comprising said salts, or crystalline forms thereof; and methods for uses of these salts, or crystalline forms thereof, for the treatment of a variety of diseases, disorders or conditions as described herein.

The present invention shall be more fully discussed with the aid of the following figures and detailed description below.

### BRIEF DESCRIPTION OF THE DRAWINGS

FIG. 1 is a powder X-ray diffractogram of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide hemi-L-tartrate hydrate.

FIG. 2 is a differential scanning calorimetry (DSC) profile for (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide hemi-L-tartrate hydrate.

FIG. 3 is a thermal gravimetric analysis (TGA) profile for (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide hemi-L-tartrate hydrate.

FIG. 4 is a vapor sorption profile (VSP) for (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide hemi-L-tartrate hydrate.

FIG. 5 is a powder X-ray diffractogram of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide mono-hydrochloride.

FIG. 6 is a differential scanning calorimetry (DSC) profile of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide mono-hydrochloride.

FIG. 7 is a thermal gravimetric analysis (TGA) profile of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide mono-hydrochloride.

FIG. 8 is a vapor sorption profile (VSP) of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide mono-hydrochloride.

FIG. 9 is a powder X-ray diffractogram of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide malonate.

FIG. 10 is a differential scanning calorimetry (DSC)/thermal gravimetric analysis (TGA) profile for (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide malonate.

FIG. 11 is a vapor sorption profile (VSP) for (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide malonate.

FIG. 12 is a powder X-ray diffractogram of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide p-toluenesulfonate hydrate.

FIG. 13 is a differential scanning calorimetry (DSC)/thermal gravimetric analysis (TGA) profile for (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide p-toluenesulfonate hydrate.

### DETAILED DESCRIPTION OF THE INVENTION

### Definitions and Abbreviations

As used above, and throughout the description of the invention, the following terms, unless otherwise indicated, shall be understood to have the following meanings.

“Tartrate Salt” is meant to describe the hemi-L-tartrate hydrate salt of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide, and has the structure of formula (IIa).

“Mono-Hydrochloride Salt” or “Mono-HCl Salt” is meant to describe the mono-hydrochloride salt of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide, and has the structure of formula (IIb).

“Malonate Salt” is meant to describe the malonate salt of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide, and has the structure of formula (IIc).

“p-Toluenesulfonate Salt” is meant to describe the p-toluenesulfonate hydrate salt of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide, and has the structure of formula (IId).

As used herein, “crystalline” refers to a solid having a highly regular chemical structure. In particular, a crystalline Salt may be produced as one or more single crystalline forms of the Salt. For the purposes of this application, the terms “single crystalline form” and “polymorph” are synonymous; the terms distinguish between crystals that have different properties (e.g., different XRPD patterns, different DSC scan results). Pseudopolymorphs are typically different solvates of a material, and thus their properties differ from one another. Thus, each distinct polymorph and pseudopolymorph of the Salt is considered to be a distinct single crystalline form herein.

“Substantially crystalline” refers to Salts that may be at least a particular weight percent crystalline. Particular weight percentages are 10%, 20%, 30%, 40%, 50%, 60%, 70%, 75%, 80%, 85%, 87%, 88%, 89%, 90%, 91%, 92%, 93%, 94%, 95%, 96%, 97%, 98%, 99%, 99.5%, 99.9%, or any percentage between 10% and 100%. In some embodiments, substantially crystalline refers to Salts that are at least 70% crystalline. In other embodiments, substantially crystalline refers to Salts that are at least 90% crystalline.

The term “solvate or solvated” means a physical association of a compound of this invention with one or more solvent molecules. This physical association includes hydrogen bonding. In certain instances the solvate will be capable of isolation, for example when one or more solvent molecules are incorporated in the crystal lattice of the crystalline solid. “Solvate or solvated” encompasses both solution-phase and isolable solvates. Representative solvates include, for example, a hydrate, ethanolates or a methanolate.

The term “hydrate” is a solvate wherein the solvent molecule is H₂O that is present in a defined stoichiometric amount, and may for example, include hemihydrate, monohydrate, dihydrate, or trihydrate.

The term “mixture” is used to refer to the combined elements of the mixture regardless of the phase-state of the combination (e.g., liquid or liquid/crystalline).

The term “seeding” is used to refer to the addition of a crystalline material to initiate recrystallization.

A “subject” is preferably a bird or mammal, such as a human, but can also be an animal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

“Treating” or “treatment” means prevention, partial alleviation, or cure of the disease. The compound and compositions of this invention are useful in treating conditions that are characterized by the activation of NF-κB and/or enhanced levels of cytokines and mediators that are regulated by NF-κB including, but not limited to TNFα and IL-1β. Inhibition or suppression of NF-κB and/or NF-κB-regulated genes such as TNFα may occur locally, for example, within certain tissues of the subject, or more extensively throughout the subject being treated for such a disease. Inhibition or suppression of NF-κB and/or NF-κB-regulated genes such as TNFα may occur by one or more mechanisms, e.g., by inhibiting or suppressing any step of the pathway(s) such as inhibition of IKK.

The term “NF-κB-associated condition” refers to diseases that are characterized by activation of NF-κB in the cytoplasm (e.g., upon phosphorylation of IκB).

The term “TNFα-associated condition” is a condition characterized by enhanced levels of TNFα. In the instant specification, the term NF-κB-associated condition will include a TNFα-associated condition, but is not limited thereto as NF-κB is involved in the activity and upregulation of other pro-inflammatory proteins and genes.

The term “inflammatory or immune diseases or disorders” is used herein to encompass both NF-κB-associated conditions and TNFα-associated conditions, e.g., any condition, disease, or disorder that is associated with release of NF-κB and/or enhanced levels of TNFα, including conditions as described herein.

“Pharmaceutically effective amount” is meant to describe an amount of a compound, composition, medicament or other active ingredient effective in producing the desired therapeutic effect.

In one aspect, the present invention is directed to Salts of the compound (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide. Accordingly, the present invention provides compounds of structural formulas (IIa), (IIb), (IIc) and (IId):

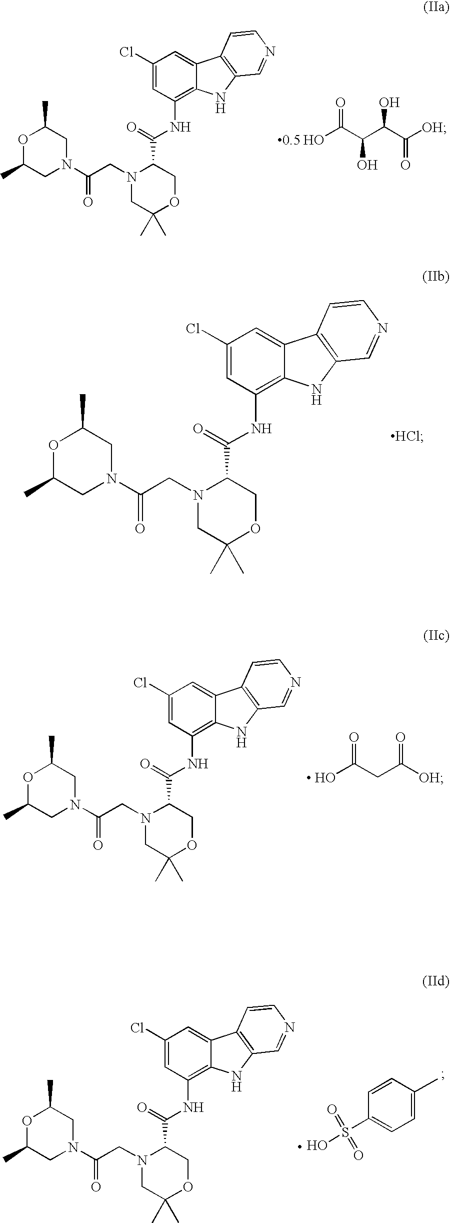

or solvates thereof.

Provided herein is an assortment of characterizing information to describe Salt forms of the compound (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide. It should be understood, however, that not all such information is required for one skilled in the art to determine that such particular form is present in a given composition, but that the determination of a particular form can be achieved using any portion of the characterizing information that one skilled in the art would recognize as sufficient for establishing the presence of a particular form, e.g., even a single distinguishing peak can be sufficient for one skilled in the art to appreciate that such particular form is present.

Surprisingly, the compounds of formula (IIa) and (IIb) exhibit considerably increased aqueous solubility over the free base form. For example, in water the crystalline free base has a solubility of about 10 μg/mL, the Tartrate Salt (IIa) has a solubility of about 0.72 mg/mL, and the Mono-Hydrochloride Salt (IIb) has a solubility of about 25 mg/mL.

In some embodiments, the Salts are substantially crystalline. Non-limiting examples of crystalline Salts include a single crystalline form of the Salt or a mixture of different single crystalline forms. An embodiment of the invention is also directed to a Salt that excludes one or more designated single crystalline forms from a particular weight percentage of Salt. Particular weight percentages may be 10%, 20%, 30%, 40%, 50%, 60%, 70%, 75%, 80%, 85%, 87%, 88%, 89%, 90%, 91%, 92%, 93%, 94%, 95%, 96%, 97%, 98%, 99%, 99.5%, 99.9%, or any percentage between 10% and 100%.

Alternatively, embodiments of the invention are directed to a crystalline Salt, wherein at least a particular percentage by weight of the crystalline Salt is a specific single crystalline form, a combination of particular crystalline forms, or excludes one or more particular crystalline forms. Particular weight percentages may be 10%, 20%, 30%, 40%, 50%, 60%, 70%, 75%, 80%, 85%, 87%, 88%, 89%, 90%, 91%, 92%, 93%, 94%, 95%, 96%, 97%, 98%, 99%, 99.5%, 99.9%, or any percentage between 10% and 100%.

Other embodiments of the invention are directed to the Salt being a single crystalline form, or being substantially a designated single crystalline form. The single crystalline form may be a particular percentage by weight of the Salt. Particular weight percentages are 10%, 20%, 30%, 40%, 50%, 60%, 70%, 75%, 80%, 85%, 87%, 88%, 89%, 90%, 91%, 92%, 93%, 94%, 95%, 96%, 97%, 98%, 99%, 99.5%, 99.9%, or any percentage between 10% and 100%. When a particular percentage by weight of a Salt is a single crystalline form, the remainder of the Salt is some combination of amorphous form of the Salt, and one or more crystalline forms of the Salt excluding the single crystalline form.

Examples of a single crystalline form include the Mono-Hydrochloride Salt, the Tartrate Salt, the Malonate Salt, and the p-Toluenesulfonate Salt, as well as descriptions of single crystalline forms characterized by one or more properties as discussed herein. The descriptions characterizing the single crystalline forms may also be used to describe the mixture of different forms that may be present in a crystalline Salt.

In the following description of particular Salts of the compound of formula (I), (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide, embodiments of the invention may be described with reference to a particular crystalline “Form” of a Salt. However, the particular crystalline forms of each Salt may also be characterized by one or more of the characteristics of the polymorph as described herein, with or without regard to referencing a particular “Form”.

Tartrate Salt (IIa)

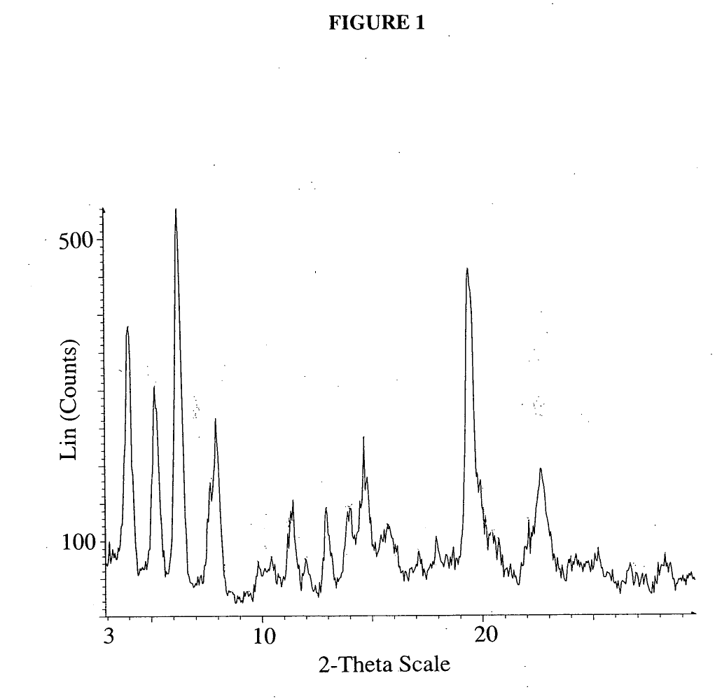

In one embodiment of the invention, a single crystalline form of the Tartrate Salt of formula (IIa) is characterized by the X-ray powder diffraction (XRPD) pattern shown in FIG. 1, and data shown in Table 1, obtained using CuKα radiation. In a particular embodiment of the invention, the polymorph can be characterized by one or more of the peaks taken from FIG. 1.

TABLE 1

| | Relative |
|---|---|
| Angle | Intensity |
| 2-θ ° | % |
| 3.970 | 70.80 |
| 5.163 | 56.50 |
| 6.203 | 100.00 |
| 7.600 | 32.70 |
| 7.939 | 48.30 |
| 8.600 | 6.30 |
| 9.372 | 5.90 |
| 9.820 | 12.90 |
| 10.405 | 14.20 |
| 11.200 | 21.80 |
| 11.349 | 28.60 |
| 11.961 | 13.50 |
| 12.946 | 26.40 |
| 13.978 | 26.20 |
| 14.645 | 43.40 |
| 15.740 | 21.80 |
| 17.152 | 15.50 |
| 17.917 | 19.20 |
| 19.451 | 85.20 |
| 20.364 | 20.50 |
| 20.740 | 18.60 |
| 22.100 | 23.10 |
| 22.676 | 36.00 |
| 23.917 | 13.10 |
| 25.232 | 16.10 |
| 26.712 | 12.40 |
| 26.940 | 10.00 |
| 27.318 | 10.00 |
| 28.281 | 14.90 |

In another embodiment of the invention, the peaks are identified at 2θ angles of 3.970°, 5.163°, 6.203°, 7.600°, 7.939°, 14.645°, 19.451°, and 22.676°. In a further particular embodiment, the peaks are identified at 2θ angles of 3.970°, 5.163°, 6.203°, and 19.451°.

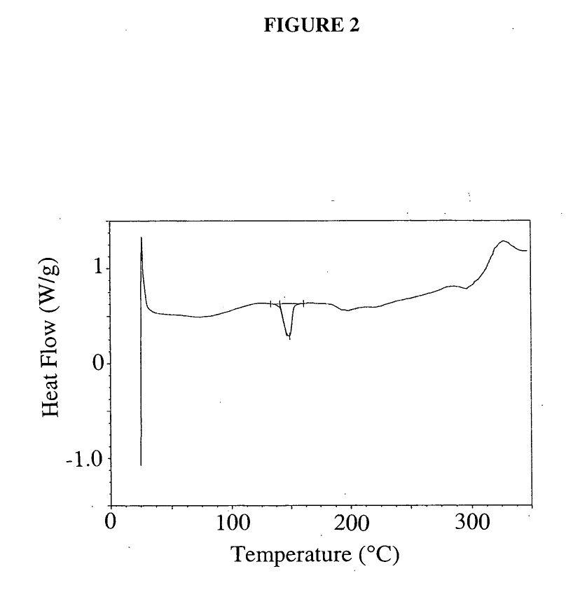

In another embodiment of the invention, the Tartrate Salt of formula (IIa) can be characterized by the differential scanning calorimetry profile (DSC) shown in FIG. 2. This Salt is hydrated, containing 3-4 water molecules; therefore it is difficult to acquire a sharp endotherm. The onset temperature is 140.8° C. with a melt of 149.0° C. These temperatures have an error of ±1° C., and are conducted at a temperature scanning rate of 10° C./minute.

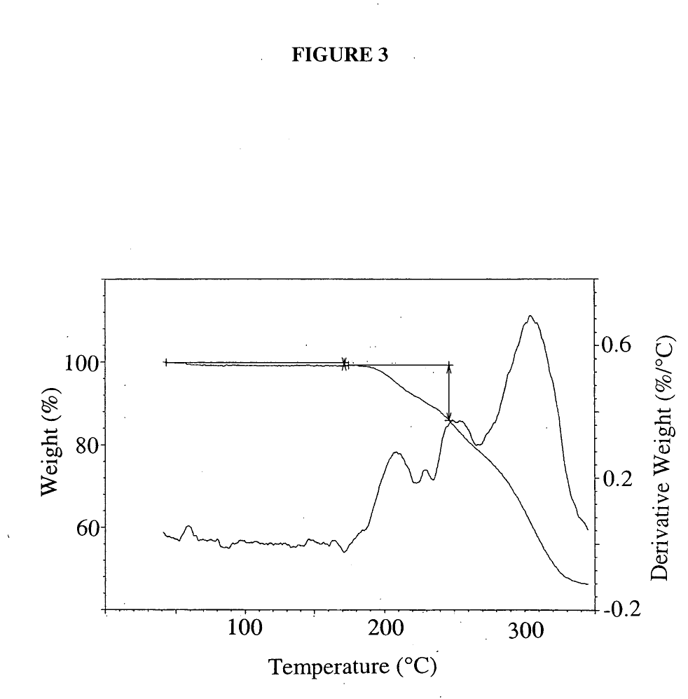

In another embodiment of the invention, the Tartrate Salt can be characterized by the thermal gravimetric analysis (TGA) profile shown in FIG. 3. The profile graphs the percent loss of weight of the sample as a function of temperature, the temperature rate change being about 10° C./min. The weight loss represents a loss of about 0.7547% of the weight of the sample as the temperature is changed from 50° C. to 170° C. The small weight loss corresponds with the weak endotherm seen in the differential scanning calorimetry (DSC) profile in FIG. 2.

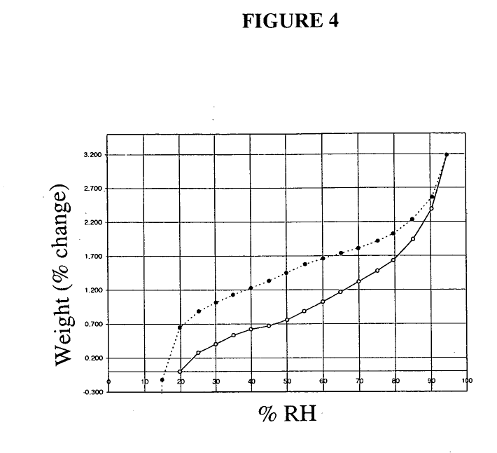

Another embodiment of the invention utilizes a vapor sorption profiles (GVS), as shown in FIG. 4 to characterize a sample of the Tartrate Salt. The profile shows the change in weight of the sample as the relative humidity of the environment is changed between 20% and 95% at a temperature of 25° C. The tartrate salt is relatively non-hygroscopic with an uptake of 1.3% at 70% RH and 2.4% at 90% RH. Hysteresis occurs and the weight gain is not completely reversible and the moisture remaining does not correlate to stoichiometric amounts of water.

In another embodiment of the invention, a single crystalline form of the Tartrate Salt is characterized by at least one of the following features (a-i)-(a-iii):
(a-i) at least one of the X-ray powder diffraction peaks shown in Table 1.
(a-ii) an X-ray powder diffraction pattern substantially similar to FIG. 1.
(a-iii) a differential scanning calorimetry (DSC) profile having an endotherm range of about 130° C. to about 160° C.

In a further embodiment of the invention a single crystalline form of the Tartrate Salt is characterized by all of the features (a-i)-(a-iii).

Mono Hydrochloride Salt (IIb)

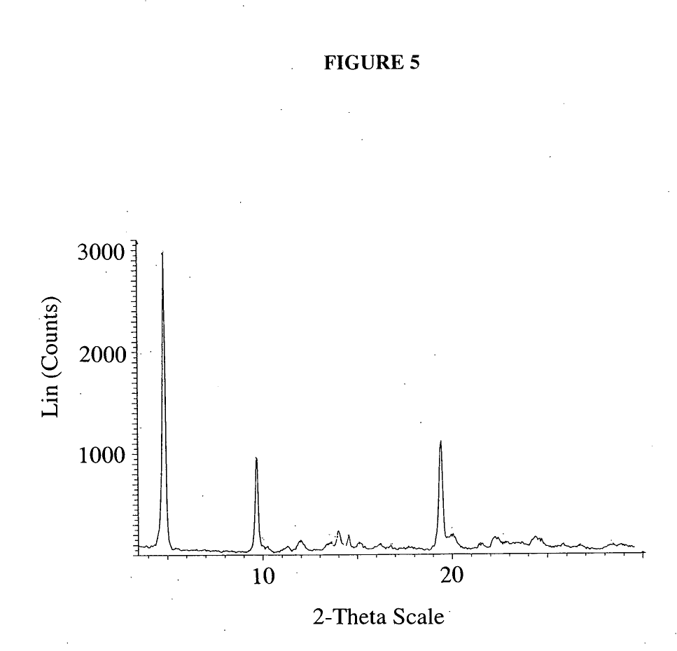

In another embodiment of the invention, a single crystalline form of the Mono-Hydrochloride Salt of formula (IIb) is characterized by the X-ray powder diffraction (XRPD) pattern shown in FIG. 5, and data shown in Table 2, obtained using CuKα radiation. In a particular embodiment of the invention, the polymorph can be characterized by one or more of the peaks taken from FIG. 5.

TABLE 2

| | Relative |
|---|---|
| Angle | Intensity |
| 2-θ° | % |
| 4.809 | 100.00 |
| 9.687 | 32.00 |
| 10.250 | 2.50 |
| 11.283 | 2.70 |
| 11.958 | 4.60 |
| 13.400 | 3.20 |
| 13.581 | 3.70 |
| 13.979 | 7.60 |
| 14.495 | 6.20 |
| 15.096 | 3.70 |
| 16.203 | 3.40 |
| 16.789 | 2.70 |
| 19.440 | 37.90 |
| 19.985 | 6.70 |
| 21.478 | 3.30 |
| 22.418 | 5.30 |
| 22.900 | 3.90 |
| 24.339 | 5.40 |
| 24.700 | 4.60 |
| 25.800 | 3.20 |
| 26.675 | 2.30 |
| 28.464 | 2.90 |

In a further particular embodiment of the invention, the peaks are identified at 2θ angles of 4.809°, 9.687°, and 19.440°.

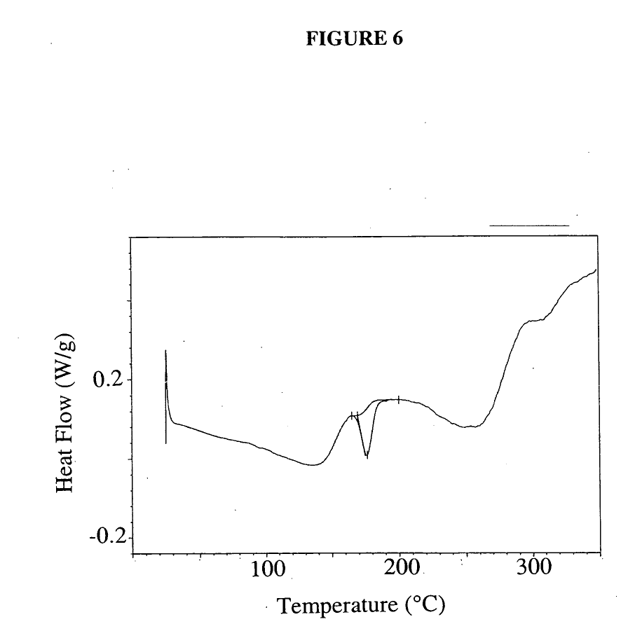

In another embodiment of the invention, the Mono-Hydrochloride Salt can be characterized by the differential scanning calorimetry (DSC) profile shown in FIG. 6. The profile plots the heat flow as a function of temperature from a sample of the Mono-Hydrochloride Salt. The profile can be characterized by one broad endotherm which has an onset temperature of 169.1° C. and a melt of 176.6° C. These temperatures have an error of ±1° C., and are conducted at a temperature scanning rate of 10° C./minute.

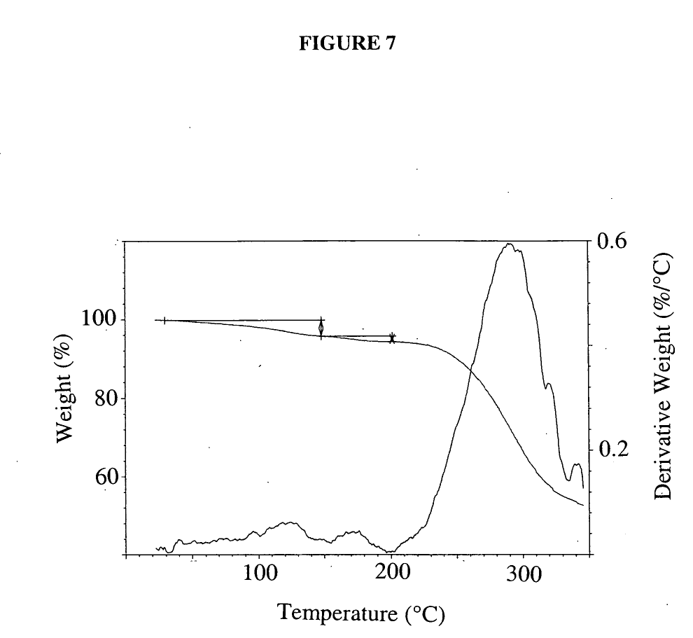

In another embodiment of the invention, the Mono-Hydrochloride Salt can also be characterized by the thermal gravimetric analysis (TGA) profile shown in FIG. 7. The profile graphs the percent loss of weight of the sample as a function of temperature, the temperature rate change being about 10° C./min. The weight loss represents a loss of about 5.500% of the weight of the sample as the temperature is changed from 100° C. to 225° C. These temperatures have an error of ±1° C.

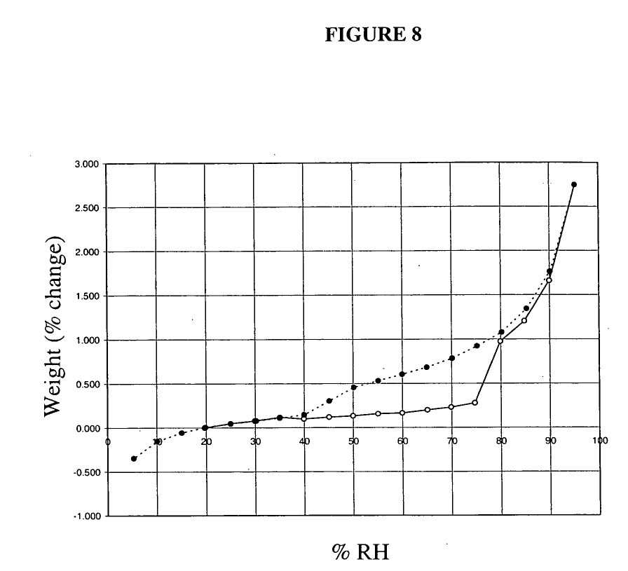

Another embodiment of the invention utilizes the vapor sorption profiles (GVS), as shown in FIG. 8, to characterize a sample of the Mono-Hydrochloride Salt. The profile shows the change in weight of a sample as the relative humidity (RH) of the environment is changed between 5% and 95% at a temperature of 25° C. The Mono-Hydrochloride Salt is relatively non-hygroscopic with moisture uptake of 0.23% at 70% RH and 1.7% at 90% RH. A slight hysteresis was observed, but the weight gain was reversible.

In another embodiment, a single crystalline form of the Mono-Hydrochloride Salt is characterized by at least one of the following features (b-i)-(b-iii):
(b-i) at least one of the X-ray powder diffraction peaks shown in Table 2.
(b-ii) an X-ray powder diffraction pattern substantially similar to FIG. 5.
(b-iii) a differential scanning calorimetry (DSC) profile having an endotherm range of about 160° C. to about 200° C.

In a further embodiment of the invention, a single crystalline form of the Mono-Hydrochloride Salt is characterized by all of the features (b-i)-(b-iii).

Malonate (IIc)

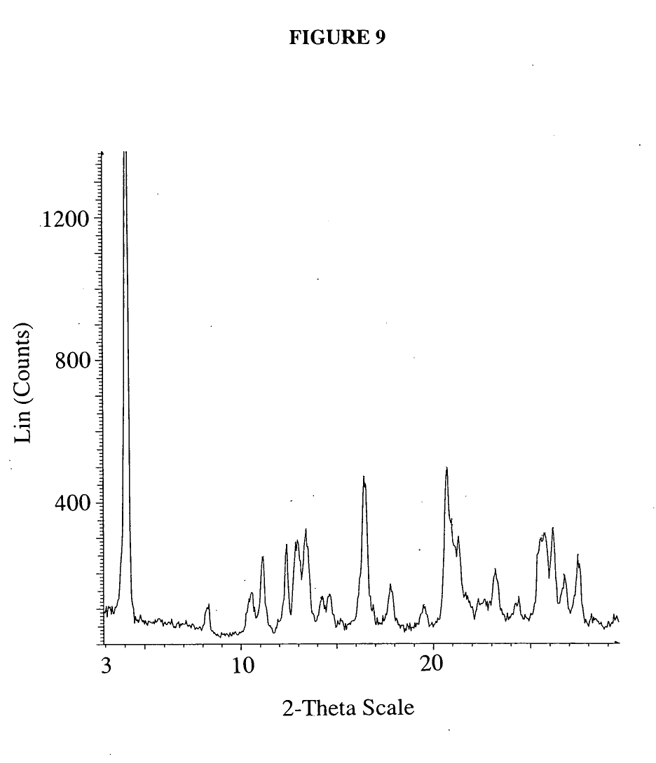

In another embodiment of the invention, a single crystalline form of the Malonate Salt of formula (IIc) is characterized by the X-ray powder diffraction (XRPD) pattern shown in FIG. 9, and data shown in Table 3, obtained using CuKα radiation. In a particular embodiment of the invention, the polymorph is characterized by one or more of the peaks taken from FIG. 9.

TABLE 3

| | Relative |
|---|---|
| Angle | Intensity |
| 2-θ ° | % |
| 4.098 | 100 |
| 8.245 | 6.8 |
| 10.516 | 8.9 |
| 11.125 | 14.7 |
| 12.401 | 17 |
| 12.935 | 17.7 |
| 13.387 | 19.7 |
| 14.204 | 7.5 |
| 14.622 | 8.3 |
| 16.473 | 28.5 |
| 17.818 | 10.3 |
| 19.532 | 6.5 |
| 20.764 | 30.2 |
| 21.346 | 18.4 |
| 23.22 | 12.7 |
| 24.409 | 7.9 |
| 25.622 | 17.8 |
| 25.788 | 18.7 |
| 26.235 | 19.6 |
| 26.82 | 11.4 |
| 27.548 | 15.1 |

In a further particular embodiment, the peaks are identified at 2θ angles of 4.098°, 16.473°, and 20.764°.

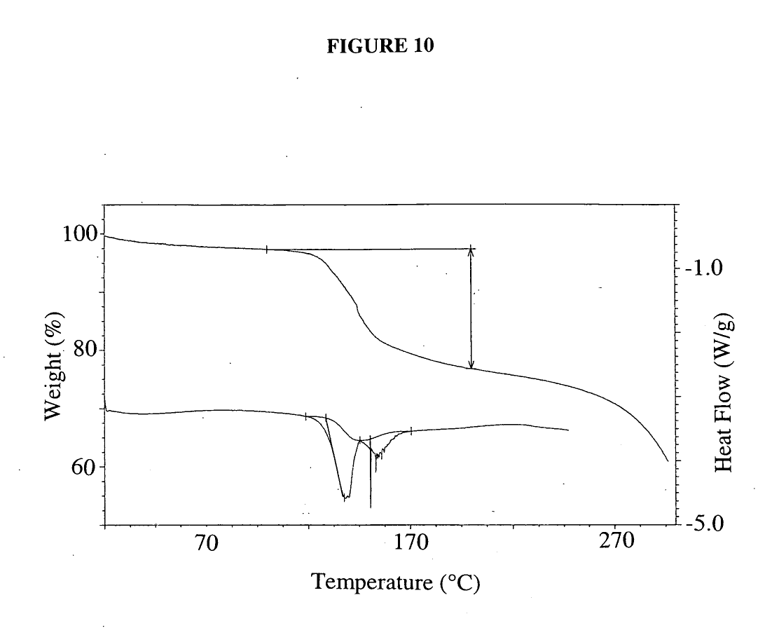

In another embodiment of the invention, the Malonate Salt can be characterized by the DSC/TGA profile shown in FIG. 10. The DSC graph plots the heat flow as a function of temperature from a sample, the temperature rate change being about 10° C./min. The profile is characterized by an endothermic transition with an onset temperature of 128.6° C. with a melt of 137.7° C. A second endothermic transition corresponding to decomposition has an onset temperature of 146° C. These temperatures have an error of ±1° C.

The Malonate Salt can also be characterized by the TGA profile also shown in FIG. 10. The profile graphs the percent loss of weight of the sample as a function of temperature, the temperature rate change being about 10° C./min. The weight loss represents a loss of about 20.57% of the weight of the sample as the temperature is changed from 50° C. to 200° C. This weight loss corresponds to loss of the malonic acid. These temperatures have an error of ±1° C.

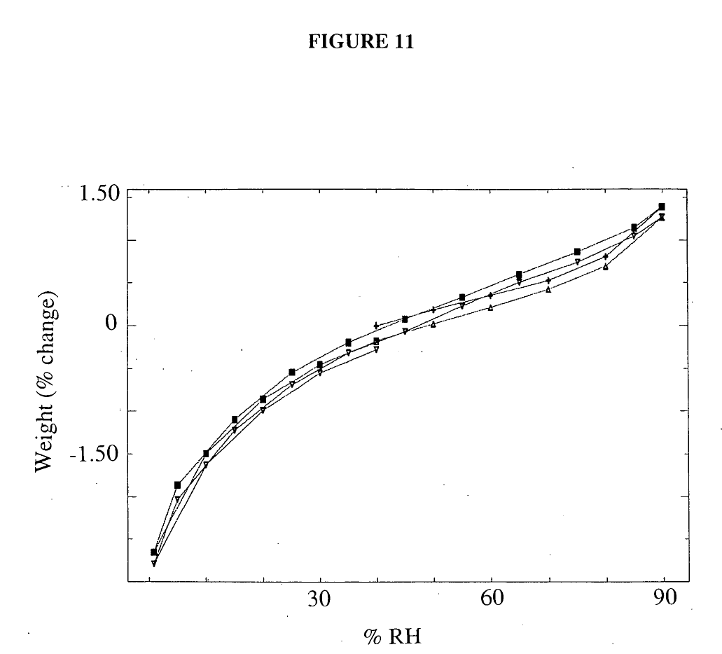

Another embodiment of the invention utilizes the vapor sorption profiles (GVS), as shown in FIG. 11 to characterize a sample of the Malonate Salt. The profiles show the change in weight of a sample of the Malonate Salt as the relative humidity (RH) of the environment is changed between 5% and 95% at a temperature of 25° C. The Malonate Salt is relatively non-hygroscopic with an uptake of 1.5 wt % from 40-90% RH. A slight hysteresis was observed, but the weight gain was reversible.

In another embodiment of the invention, a single crystalline from of the Malonate Salt is characterized by at least one of the following features (c-i)-(c-iii):
(c-i) at least one of the X-ray powder diffraction peaks shown in Table 4.
(c-ii) an X-ray powder diffraction pattern substantially similar to FIG. 9.
(c-iii) a differential scanning calorimetry (DSC) profile having an endotherm range of about 115° C. to about 170° C.

In a further embodiment of the invention, a single crystalline form of the Malonate Salt is characterized by all of features (c-i)-(c-iii).

p-Toluenesulfonate (IId)

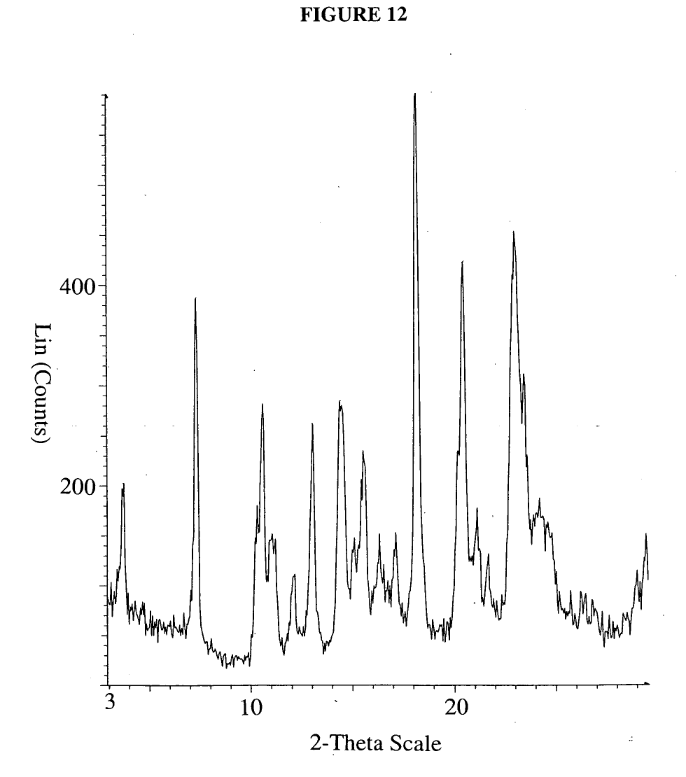

In another embodiment of the invention, a single crystalline form of the p-Toluenesulfonate Salt of formula (IId) is characterized by the X-ray powder diffraction (XRPD) pattern shown in FIG. 12, and data shown in Table 4, obtained using CuKα radiation. In a particular embodiment of the invention, the polymorph is characterized by one or more of the peaks taken from FIG. 12.

TABLE 4

| | Relative |
|---|---|
| Angle | Intensity |
| 2-θ ° | % |
| 3.646 | 34.1 |
| 7.293 | 65.4 |
| 10.574 | 47.6 |
| 11.103 | 25.7 |
| 12.081 | 18.9 |
| 13.041 | 44.3 |
| 14.451 | 48.1 |
| 15.591 | 39.7 |
| 17.121 | 25.7 |
| 18.236 | 100 |
| 20.488 | 71.3 |
| 23.081 | 76.7 |

In a further particular embodiment, the peaks are identified at 2θ angles of 3.646°, 7.293°, 10.574°, 13.041°, 14.451°, 15.591°, 18.236°, 20.488°, and 23.081°. In another further particular embodiment, the peaks are identified at 2θ angles of 7.293°, 18.236°, 20.488°, and 23.081°.

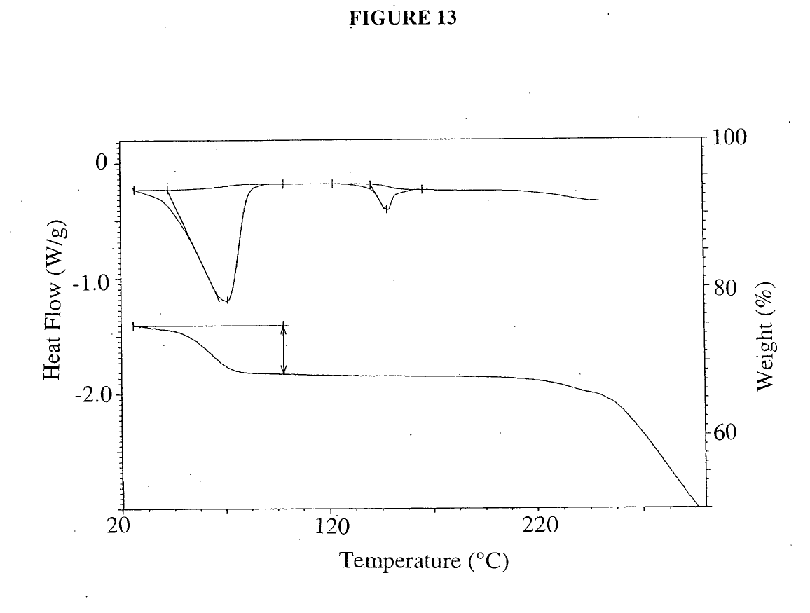

In another embodiment of the invention, the p-Toluenesulfonate Salt can be characterized by the DSC/TGA profile shown in FIG. 13. The DSC graph plots the heat flow as a function of temperature from a sample, the temperature rate change being about 10° C./min. The DSC profile is characterized by a broad endotherm with an onset temperature of 42.4° C. with a maximum at 71.2° C. corresponding to the loss of water in the TGA profile. The profile is also characterized by a weak endotherm with an onset of 140.5° C. and melt of 148.2° C. These temperatures have an error of ±1° C.

The p-Toluenesulfonate Salt can also be characterized by the TGA profile also shown in FIG. 13. The profile graphs the percent loss of weight of the sample as a function of temperature, the temperature rate change being about 10° C./min. The weight loss represents a loss of about 6.59% of the weight of the sample as the temperature is changed from 20° C. to 90° C. This weight loss corresponds to the loss of about 1 mole of water. These temperatures have an error of ±1° C.

In another embodiment of the invention, a single crystalline form of the p-Toluenesulfonate Salt is characterized by at least one of the following features (d-i)-(d-iv):
(d-i) at least one of the X-ray powder diffraction peaks shown in Table 5.
(d-ii) an X-ray powder diffraction pattern substantially similar to FIG. 12.
(d-iii) a differential scanning calorimetry (DSC) profile having a first endotherm range of about 25° C. to about 105° C.
(d-iv) a differential scanning calorimetry (DSC) profile having a second endotherm range of about 130° C. to about 165° C.

In a further embodiment of the invention, a single crystalline form of the p-Toluenesulfonate Salt is characterized by all of features (d-i)-(d-iv).

Pharmaceutical Compositions and Methods

The pharmacological properties of any of the compounds of formula (IIa), (IIb), (IIc), (IId), or crystalline forms thereof, are such that it is suitable for use in the treatment of all those patients suffering from or subject to conditions that can be ameliorated by the administration of an inhibitor of IκB kinase.

In yet another aspect, a method for treating an inflammatory disease or immune-related disease is provided comprising administering a pharmaceutically effective amount of any of the compounds of formula (IIa), (IIb), (IIc) or (IId), including crystalline forms thereof, or a pharmaceutical composition thereof, to a subject in need thereof. In still another aspect, a method for treating cancer is provided comprising administering a pharmaceutically effective amount of any of the compounds of formula (IIa), (IIb), (IIc) or (IId), including crystalline forms thereof, or a pharmaceutical composition thereof, to a subject in need thereof.

More particularly, the present compounds are useful for treating or lessening the severity of an inflammatory disease, an immune-related disease or cancer. In some embodiments, these diseases and disorders include, but are not limited to, joint inflammation (e.g., rheumatoid arthritis (RA), rheumatoid spondylitis, gouty arthritis, traumatic arthritis, rubella arthritis, psoriatic arthritis, osteoarthritis, and other arthritic conditions), acute synovitis, tuberculosis, atherosclerosis, muscle degeneration, cachexia, Reiter's syndrome, endotoxaemia, sepsis, septic shock, endotoxic shock, gram negative sepsis, gout, toxic shock syndrome, pulmonary inflammatory diseases (e.g., asthma, acute respiratory distress syndrome, chronic obstructive pulmonary disease, silicosis, pulmonary sarcoidosis, and the like), bone resorption diseases, reperfusion injuries, carcinoses, leukemia, sarcomas, lymph node tumors, skin carcinoses, lymphoma, apoptosis, graft versus host reaction, graft versus host disease (GVHD), allograft rejection, leprosy, viral infections (e.g., HIV, cytomegalovirus (CMV), influenza, adenovirus, the Herpes group of viruses, and the like), parasitic infections (e.g., malaria, such as cerebral malaria), yeast and fungal-infections (e.g., fungal meningitis), fever and myalgias due to infection, acquired immune deficiency syndrome (AIDS), AIDS related complex (ARC), cachexia secondary to infection or malignancy, cachexia secondary to AIDS or cancer, keloid and scar tissue formation, pyresis, diabetes, inflammatory bowel diseases (IBD) (e.g., Crohn's disease and ulcerative colitis), multiple sclerosis (MS), ischemic brain injury, e.g. cerebral infarction (stroke), head trauma, psoriasis, Alzheimer's disease, carcinomatous disorders (potentiation of cytotoxic therapies), cardiac infarct, chronic obstructive pulmonary disease (COPD), COPD exacerbations, and acute respiratory distress syndrome (ARDS). In other embodiments, compounds of the invention are useful for treating cancer, especially for treating cancers where IKK activity is abnormally high. The cancer types that may be treated include lymphoma, such as diffuse large B-cell (Davis, et al., J. Exp. Med. 2001, 194, 1861-1874; Lam et al., Clin. Cancer Res. 2005, 11, 28-40; Feuerhake et al., Blood, 2005, 106, 1392-1399), primary mediastinal B-cell, and mantle cell; multiple myeloma (Berenson et al., Clin. Adv. Hematol. Oncol. 2004, 2, 162-166; Gunn et al., Stem Cells, 2005); osteolytic bone metastasis (Ruocco et al., J. Exp. Med. 2005, 201, 1677-1687; Morony et al., Endocrinology 2005, 146, 3235-3243; Gordon, et al., Cancer Res. 2005, 65, 3209-3217; RoleSohara et al., Cancer Lett. 2005, 228, 203-209); head and neck squamous cell cancer (van Hogerlinden et al., J. Invest. Dermatol. 2004, 123 101-108; Tamatani et al, Int. J. Cancer. 2004, 108, 912-921; Loercher et al., Cancer Res. 2004, 64, 6511-6523; Van Waes et al., Int. J. Radiat. Oncol. Biol. Phys. 2005, 63, 1400-1412); prostate cancer; pancreatic cancer and non-small cell lung cancer. In some embodiments, any of the compounds of formula (IIa), (IIb), (IIc) or (IId), or crystalline forms thereof, is useful for treating inflammatory and immune-related diseases, disorders and symptoms, more especially, inflammatory ones such as RA, asthma, IBD, psoriasis, psoriatic arthritis, COPD, COPD exacerbations and MS. In some embodiments, any of the compounds of formula (IIa), (IIb), (IIc) or (IId), or crystalline forms thereof, is useful for treating inflammatory and immune-related diseases, disorders and symptoms, more especially, inflammatory ones such as RA, IBD, psoriasis, COPD and COPD exacerbations. In a further embodiment, any of the compounds of formula (IIa), (IIb), (IIc) or (IId), or crystalline forms thereof, is useful for treating inflammatory and immune-related diseases, disorders and symptoms, more especially, inflammatory ones such as RA.

It will also be appreciated that any of the compounds of formula (IIa), (IIb), (IIc) or (IId), or crystalline forms thereof, are useful for treating diseases, disorders or symptoms related to the activity of NF-κB, TNF-α, and other enzymes in pathways where IKK is known to modulate activity.

Accordingly, in another aspect of the present invention, pharmaceutical compositions are provided, wherein these compositions comprise any of the compounds of formula (IIa), (IIb), (IIc) or (IId), or crystalline forms thereof, and a pharmaceutically acceptable carrier. In certain embodiments, these compositions optionally further comprise one or more additional therapeutic agents.

As described above, the pharmaceutically acceptable compositions of the present invention additionally comprise a pharmaceutically acceptable carrier, which, as used herein, includes any and all solvents, diluents, or other liquid vehicle, dispersion or suspension aids, gelatin or polymeric capsule shell, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Any of the compounds of formula (IIa), (IIb), (IIc) or (IId), or crystalline forms thereof, or a pharmaceutical composition thereof, according to the method of the present invention, may be administered using any amount and any route of administration effective for treating the disease. The exact amount required will vary from subject to subject, depending on the species, age, and general condition of the subject, the severity of the infection, the particular agent, its mode of administration, and the like. Any of the compounds of formula (IIa), (IIb), (IIc) or (IId), or crystalline forms thereof, or a pharmaceutical composition thereof, are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disease being treated and the severity of the disease; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Any of the compounds of formula (IIa), (IIb), (IIc) or (IId), or crystalline forms thereof, or a pharmaceutical composition thereof, can be administered to humans and other animals orally, rectally, parenterally, intracistemally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound. Alternatively, compositions for rectal or vaginal administration are gels or creams that can be prepared by mixing compounds with suitable non-irritating excipients such as oils or water to solubilize the compound and polymers and fatty alcohols can be added to thicken the formulation to increase the residual time in the rectal or vaginal cavity and release the active compound.

Solid dosage forms for oral administration include capsules, tablets, pills, powders, and granules. In such solid dosage forms, the active compound may optionally be mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. In other embodiments, the active compound may be encapsulated in a gelatin or polymeric capsule shell without any additional agents (neat capsule shell).

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. The solid dosage forms may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

While any of the compounds of formula (IIa), (IIb), (IIc) or (IId), or crystalline forms thereof, may be used in an application of monotherapy to treat a disorder, disease or symptom, it also may be used in combination therapy, in which the use of an inventive compound or composition (therapeutic agent) is combined with the use of one or more other therapeutic agents for treating the same and/or other types of disorders, symptoms and diseases. Combination therapy includes administration of the therapeutic agents concurrently or sequentially. Alternatively, the therapeutic agents can be combined into one composition which is administered to the patient.

In one embodiment, any of the compounds of formula (IIa), (IIb), (IIc) or (IId), or crystalline forms thereof, is used in combination with other therapeutic agents, such as other inhibitors of IKK, other agents useful in treating NF-κB and TNF-α associated conditions, and agents useful for treating other disorders, symptoms and diseases. In particular, agents that induce apoptosis such as agents that disrupt cell cycle or mitochondrial function are useful in combination with the IKK inhibitors of this invention. Exemplary agents for combination with the IKK inhibitors include antiproliferative agents (e.g., methotrexate) and the agents disclosed in U.S. Pat. Application Publication No. US2003/0022898, p 14, para. [0173-0174], which is incorporated herein in its entirety. In some embodiments, the compound of the invention is administered in conjunction with a therapeutic agent selected from the group consisting of cytotoxic agents, radiotherapy, and immunotherapy. Non-limiting examples of cytotoxic agents suitable for use in combination with the IKK inhibitors of the invention include capecitibine; gemcitabine; irinotecan; fludarabine; 5-fluorouracil or 5-fluorouracil/leucovorin; taxanes, including, e.g., paclitaxel and docetaxel; platinum agents, including, e.g., cisplatin, carboplatin, and oxaliplatin; anthracyclins, including, e.g., doxorubicin and pegylated liposomal doxorubicin; mitoxantrone; dexamethasone; vincristine; etoposide; prednisone; thalidomide; herceptin; temozolomide; and alkylating agents such as melphalan, chlorambucil, and cydophosphamide. It is understood that other combinations may be undertaken while remaining within the scope of the invention.

The preparation and properties of the compounds of the invention are described in the following experimental section.

### EXAMPLES

### Example 1

Preparation of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide hemi-L-tartrate hydrate (IIa): A reaction vessel was charged with (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide (4.00 g, 7.78 mmol), L-tartaric acid (1.2 g, 7.8 mmol), water (64 mL) and acetone (32 mL). This slurry was heated to 50° C. where it rapidly became homogeneous. The solution was seeded between 45-50° C. and held at 45° C. for 1 h. The slurry was allowed to cool to ambient temperature and was isolated by filtration with 2:1 water:acetone wash (5 mL) to provide (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide hemi-L-tartrate hydrate (3.84 g) after drying.

### Example 2

Preparation of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide mono-hydrochloride (IIb): A reaction vessel was charged with (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide (300 mg, 0.58 mmol) and ethyl acetate (0.15 mL). The mixture was heated at 55° C. to dissolve the solids and then charged with 5-6 N HCl in isopropanol (0.117 mL, 0.58 mmol, 1 equiv.). The solution was allowed to stir at 55° C. for 10 mins before the addition of toluene (6 mL). The solution was seeded before the addition of toluene (3 mL). The mixture was cooled to ambient temperature and allowed to stir overnight. The resulting slurry was cooled in an ice bath for 3 h before isolation by filtration to provide (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide mono-hydrochloride (38 mg, 12%) after drying.

### Example 3

Preparation of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide malonate (IIc): A reaction vessel was charged with (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide (1.05 g, 2.04 mmol) and acetonitrile (10 mL). To this solution was charged a 1 M solution of malonic acid in acetone (2.05 mL, 2.05 mol). After 1 h the resulting precipitate was collected by filtration to provide (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide malonate (0.90 g, 71%).

A similar procedure can be conducted in acetone instead of acetonitrile, and also provides (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide malonate (IIc).

### Example 4

Preparation of (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide p-toluenesulfonate hydrate (IId): A reaction vessel was charged with amorphous (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide p-toluenesulfonate (0.70 g, 0.10 mmol) and water (700 μL) and allowed to stir overnight at ambient temperature. The material was isolated by filtration to provide (S)—N-(6-chloro-9H-pyrido[3,4-b]indol-8-yl)-4-(2-((2S,6R)-2,6-dimethylmorpholino)-2-oxoethyl)-6,6-dimethylmorpholine-3-carboxamide p-toluenesulfonate hydrate after drying.

### Example 5

Solubility: The water solubility of the Tartrate and Mono-HCl Salt was measured at ambient temperature. Table 5 is a summary of the equilibrium solubility. For the Tartrate and Mono-HCl Salts, the solubility is much greater than the free base which has an intrinsic solubility of ˜10 μg/mL.

TABLE 5

| Salt | Solubility (mg/mL)* | pH |
|---|---|---|
| L-Tartrate (IIa) | 0.72 | 4.96 |
| Mono-HCl (IIb) | 25 | 3.71 |

*expressed as free base

### Example 6

X-Ray Powder Diffractometry (XRPD): X-ray powder diffraction patterns for the samples were acquired on a Bruker AXS D8Advance diffractometer. The data are collected over an angular range of 2.9° to 29.6° 2θ in continuous scan mode using a step size of 0.05° 2θ and a step time of 2 seconds. The sample is run under ambient conditions and prepared as a flat plate specimen using powder as received without grinding.

### Example 7

Differential Scanning Calorimetry (DSC): Differential scanning calorimetry (DSC) data are collected on a TA Instruments Q100 differential scanning calorimeter equipped with a 50 position auto-sampler. The energy and temperature calibration standard is indium. Samples are heated at a rate of either 5° C. or 10° C. per minute between 25° C. and 300° C. A nitrogen purge flowing at 50 mL per minute is maintained over the sample during a scan. Between 1 mg and 3 mg of sample is analyzed. All samples are crimped in a hermetically sealed aluminum pan with a pinhole to alleviate the pressure accumulated from the solvent vapor.

### Example 8

Thermal Gravimetric Analysis (TGA): Thermal gravimetric analysis (TGA) data are collected on a TA Instruments Q500 thermal gravimetric analyzer, calibrated with Nickel/Alumel and running at a scan rate of either 5° C. or 10° C. per minute. A nitrogen purge flowing at 60 mL per minute is maintained over the sample during measurements. Typically 5 mg to 15 mg of sample is loaded onto a pre-tared platinum crucible.

### Example 9

Gravimetric Vapor Sorption (GVS): Gravimetric vapor sorption (GVS) data are collected using either i) a SGA-100 Water Vapor Sorption Analyzer from VTI Corporation. Sample sizes are typically 5-10 mg. A moisture adsorption/desorption isotherm is recorded by subjecting samples to a series of relative humidity (RH) steps at a constant temperature of 25° C.; or ii) a Hiden IGASorp moisture sorption analyser running CFRSorp software, IGA Systems Software V3.00.23 and IGASorp Controller Version 1.10. Sample sizes are typically 10 mg. A moisture adsorption/desorption isotherm iserformed in the following way: Samples are loaded/unloaded at typical room humidity and temperature (40% RH, 25° C.) and analysed afterwards by XRPD. The isotherm run is two complete cycles. Each cycle begins at 40% RH and using 10% RH intervals goes to 90% RH then Dry then 40% RH.

### Example 10

### Biological Testing

Compounds of this invention are effective inhibitors of IκB kinase (IKK), and therefore, are useful for treating conditions caused or aggravated by the activity of this kinase. The in vitro and in vivo IκB kinase inhibitory activities of the compounds of the invention may be determined by various procedures known in the art. The potent affinities for IκB kinase exhibited by the inventive compounds can be measured as an IC₅₀ value (in nM), which is the concentration (in nM) of compound required to provide 50% inhibition of IκB kinase.

Following are examples of assays that can be useful for evaluating and selecting a compound that modulates IKK.

Assay for Measuring IκB Kinase Enzyme Inhibition

An in vitro assay for detecting and measuring inhibition activity against IκB kinase complex by candidate pharmacological agents can employ a biotinylated GST fusion protein spanning residues 5-55 of IκBA (SwissProt Accession No. P25963, Swiss Institute of Bioinformatics, Geneva, Switzerland) and an agent for detection of the phosphorylated product, e.g. a specific antibody binding only to the phosphorylated form GS, being either monoclonal or polyclonal (e.g., commercially-available anti-phospho-serine³² IκB antibodies). In the example of detecting the phosphorylated product by an anti-phosphoserines^{32 and 36} IκB antibody, once the antibody-phospho-GST-IκBα complex is formed, the complex can be detected by a variety of analytical methods (e.g., radioactivity, luminescence, fluorescence, or optical absorbance). For the use of the time resolved fluorescence method the antibody is labeled with europium chelate and the antibody-phospho-GST-IκBα complex is bound to biotin binding protein conjugated to a fluorescence acceptor (e.g., Steptavidin Alexa647, Invitrogen, Carlsbad, Calif.). How to prepare materials for and conduct this assay are described in more detail below.

Isolation of the IκB Kinase Complex

An IκB-α kinase complex is prepared by first diluting 10 ml of HeLa S3 cell-extracts S100 fraction (Lee et al., Cell 1997, 88, 213-222) with 40 ml of 50 mM HEPES pH 7.5. Then, 40% ammonium sulfate is added and incubated on ice for 30 minutes. The resulting precipitated pellet is redissolved with 5 ml of SEC buffer (50 mM HEPES pH 7.5, 1 mM DTT, 0.5 mM EDTA, 10 mM 2-glycerophosphate), clarified by centrifugation at 20,000×g for 15 min, and filtrated through a 0.22 μm filter unit. The sample is loaded onto a 320 ml SUPEROSE-6 gel filtration FPLC column (Amersham Biosciences AB, Uppsala, Sweden) equilibrated with a SEC buffer operated at 2 ml/min flow rate at 4° C. Fractions spanning the 670-kDa molecular-weight marker are pooled for activation. A kinase-containing pool is then activated by incubation with 100 nM MEKK1Δ (Lee et al., Cell 1997, 88, 213-222) 250 μM MgATP, 10 mM MgCl₂, 5 mM DTT, 10 mM 2-glycerophosphate, 2.5 μM Microcystin-LR, for 45 minutes at 37° C. The activated enzyme is stored at −80° C. until further use.

Measurement of IκB Kinase Phospho-Transferase Activity

To each well of a 384 well plate, compounds of various concentrations in 1 μL of DMSO are incubated for 2 hours with 30 μL of assay buffer (50 mM Hepes pH 7.5, 5 mM DTT, 10 mM MgCl₂ 10 mM 2-glycerophosphate, 0.1% Bovine Serum Albumin) containing a 1:90 dilution of activated enzyme, 100 nM biotinylated-GST-IκBα 5-55, and 50 μM ATP. Reactions are quenched with the addition of 10 μL of 250 mM EDTA before the addition of 40 μL of detection buffer (50 mM Hepes pH 7.5, 0.1% Bovine Serum Albumin, 0.01% Tween20, Pierce, Rockford, Ill.) containing 2 nM europium labeled anti-IκBα phosphoserine^{32 and 36} and 0.003 mg/mL Streptavidin Alexa647. Samples are allowed to incubate for 1 hour prior to reading on a Wallac Victor plate reader (Perkin Elmer Life and Analytical Sciences, Boston, Mass.). As the assay has been previously shown to be linear with respect to enzyme concentration and time at the enzyme dilution tested, levels of time resolved fluorescence energy transfer are used to determine the inhibition activity of candidate pharmacological agents.

The compounds of the invention are inhibitors of the IKK complex. It will be appreciated that compounds of this invention can exhibit IκB kinase inhibitor activities of varying degrees. Following assay procedures described herein, the IκB kinase inhibition average IC₅₀ values for the inventive compounds were generally below about 10 micromolar, preferably below about 1.0 micromolar, and more preferably below about 100 nanomolar.

Cellular Assays: A variety of cellular assays are also useful for evaluating compounds of the invention:

Multiple Myeloma (MM) Cell Lines and Patient-Derived MM Cells Isolation

RPMI 8226 and U266 human MM cells are obtained from American Type Culture Collection (Manassas, Va.). All MM cell lines are cultured in RPMI-1640 containing 10% fetal bovine serum (FBS, Sigma-Aldrich Co., St. Louis, Mo.), 2 mM L-glutamine, 100 U/mL penicillin (Pen) and 100 μg/mL streptomycin (Strep) (GIBCO brand cell culture products available from Invitrogen Life Technologies, Carlsbad, Calif.). Patient-derived MM cells are purified from patient bone marrow (BM) aspirates using ROSETTESEP (B cell enrichment kit) separation system (StemCell Technologies, Vancouver, Canada). The purity of MM cells are confirmed by flow cytometry using PE-conjugated anti-CD138 antibody (BD Biosciences, Bedford, Mass.).

Bone Marrow Stroma Cell Cultures

Bone marrow (BM) specimens are obtained from patients with MM. Mononuclear cells (MNCs) separated by Ficoll-Hipaque density sedimentation are used to establish long-term BM cultures as previously described (Uchiyama et al., Blood 1993, 82, 3712-3720). Cells are harvested in Hank's Buffered Saline Solution (HBSS) containing 0.25% trypsin and 0.02% EDTA, washed, and collected by centrifugation.

Cell Proliferation Via Measurement of DNA-Synthesis Rate

Proliferation is measured as described (Hideshima et al., Blood 2000, 96, 2943). MM cells (3×10⁴ cells/well) are incubated in 96-well culture plates (Corning Life Sciences, Corning, N.Y.) in the presence of media or an IKK inhibitor of this invention for 48 h at 37° C. DNA synthesis is measured by [³H]-thymidine ([3H]-TdR, New England Nuclear division of Perkin Elmer Life and Analytical Sciences, Boston, Mass.) incorporation into dividing cells. Cells are pulsed with [3H]TdR (0.5 μCi/well) during the last 8 h of 48 h cultures. All experiments are performed in triplicate.

MT Cell Viability Assay

The inhibitory effect of the present compounds on MM growth is assessed by measuring the reduction of yellow tetrazolium MT (3-(4,5-dimethylthiazolyl-2)-2,5-diphenyltetrazolium bromide) by metabolically active cells (J. Immunol. Methods 1994, 174, 311-320). Cells from 48 h cultures are pulsed with 10 μL of 5 mg/mL MT to each well for the last 4 h of the 48 h cultures, followed by 100 μL isopropanol containing 0.04N HCl. Absorbance is measured at 570 nm using a spectrophotometer (Molecular Devices Corp., Sunnyvale Calif.).

NF-κB Activation Via Electrophoretic Mobility Shift Assay

Electrophoretic mobility shift analyses (EMSA) are carried out as described (Hideshima et al., Oncogene 2001, 20, 4519). Briefly, MM cells are pre-incubated with an IKK inhibitor of this invention (10 μM for 90 min) before stimulation with TNF-α (5 ng/mL) for 10 to 20 min. Cells are then pelleted, resuspended in 400 μL of hypotonic lysis buffer (20 mM HEPES, pH 7.9, 10 mM KCl, 1 mM EDTA, 0.2% Triton X-100, 1 mM Na₃VO₄, 5 mM NaF, 1 mM PMSF, 5 μg/mL leupeptin, 5 μg/mL aprotinin), and kept on ice for 20 min. After centrifugation (14000 g for 5 min) at 4° C., the nuclear pellet is extracted with 100 μL hypertonic lysis buffer (20 mM HEPES, pH 7.9, 400 mM NaCl, 1 mM EDTA, 1 mM Na₃VO₄, 5 mM NaF, 1 mM PMSF, 5 μg/mL leupeptin, 5 μg/mL aprotinin) on ice for 20 min. After centrifugation (14000 g for 5 min) at 4° C., the supernatant is collected as nuclear extract. Double-stranded NF-κB consensus oligonucleotide probe (5′-GGGGACTTTCCC-3′, Santa Cruz Biotechnology Inc., Santa Cruz Calif.) is end-labeled with [(³²P]ATP (50 μCi at 222 TBq/mM; New England Nuclear division of Perkin Elmer Life and Analytical Sciences, Boston, Mass.). Binding reactions containing 1 ng of oligonucleotide and 5 μg of nuclear protein are conducted at room temperature for 20 min in a total volume of 10 μL of binding buffer (10 mM Tris-HCl, pH 7.5, 50 mM NaCl, 1 mM MgCl₂, 0.5 mM EDTA, 0.5 mM DTT, 4% glycerol (v/v), and 0.5 μg poly (dI-dC) (Amersham Biosciences AB, Uppsala, Sweden). For supershift analysis, 1 μg of anti-p65 NF-κB Ab is added 5 min before the reaction mixtures, immediately after addition of radiolabeled probe. The samples are loaded onto a 4% polyacrylamide gel, transferred to Whatman paper (Whatman International, Maidstone, U.K.), and visualized by autoradiography.

Diffuse Large B-Cell Lymphoma (DLBCL) Cell Proliferation Assay

ABC-like (LY3 and Ly10) and GCB-like (Ly7 and Ly19) DLBCL cell lines (Alizadeh et al., Nature 2000, 403, 503-511; Davis et al., J. Exp. Med. 2001, 194, 1861-1874) are maintained in growth medium (GM, Iscove's DMEM+10% FBS) by passaging cells twice per week. Cells are starved overnight in Iscove's DMEM medium+0.5% FBS overnight before being plated in proliferation assay. On the day of the assay, cells are counted and viability is checked using Trypan Blue staining. For the Ly3 and Ly10 cells, 5000 cell are plated in GM per well in a 96-well plate. The Ly7 and Ly19 cells are plated at 10,000 cells per well. IKK inhibitors are first dissolved in DMSO and then diluted in GM to reach the final concentrations of 80 μM-0.01 μM. Each concentration is plated in triplicate. Cell viability is determined using a standard WST-1 cell viability assay (Roche Applied Science, Indianapolis, Ind.).

Human Peripheral Blood Monocyte (PBMC) Cytokine Release Assay

Human PBMC is purified from normal donor whole blood by Ficoll gradient method. After a PBS wash, PBMC are re-suspended in AIM-V medium. Serially diluted IKK inhibitors of this invention in 100% DMSO are added at 11 to the bottom of a 96-well plate and mixed with 180 μl 4.5×10⁵ PBMC in AIM-V media per well. After preincubating PBMC with inhibitor at 37° C. for 40 min, cells are stimulated with 20 μl of either LPS (100 ng/ml) or anti-CD3 (0.25 μg/ml) and anti-CD28 (0.25 μg/ml) (Pharmingen division of BD Biosciences, Bedford, Mass.) at 37° C. for 5 hours. The supernatants are collected and assessed for IL-1β or TNFα release using standard commercially available ELISA kits.

Human Chondrocyte Matrix Metalloproteases (MMPs) Release Assay

Human chondrocyte cell line SW1353 (ATCC, Manassas, Va.) is cultured containing 10% fetal bovine serum (Hyclone, Logan, Utah), 2 mM L-glutamine (GIBCO brand cell culture products available from Invitrogen Life Technologies, Carlsbad, Calif.) and 1% Pen/Strep (GIBCO). Cells are seeded in 96-well Poly-D-Lysine plate (BD BICCOAT, Black/Clear bottom, BD Biosciences, Bedford, Mass.). Serially diluted IKK inhibitors at 1 μl are added to each well of 96-well plates and mixed with 180 μl 4.5×10⁵ chondrocytes per well. After pre-incubating cells with compounds for 1 hr at 37° C., cells are stimulated with 20 μl IL-1β (10 ng/mL, R&D Systems Inc.) at 37° C. for 24 hrs. The supernatants are then collected and assessed for production of matrix metalloproteinases (MMPs) using commercially available ELISA kits.

Human Fibroblast Like Synoviocyte (HFLS) Assay

HFLS isolated from RA synovial tissues obtained at joint replacement surgery are provided by Cell Applications Inc. (San Diego, Calif.). IKK inhibitors of the invention are tested for their ability to block the TNF- or IL-1β induced release of IL-6 or IL-8 from these cells using commercially available ELISA kits. Cell culture conditions and assay methods are described in Aupperle et al., Journal of Immunology, 1999, 163, 427-433.

Human Cord Blood Derived Mast Cell Assay

Human cord blood is obtained from Cambrex (Walkersville, Md.). Mast cells are differentiated and are cultured in a manner similar to that described by Hsieh et al., J. Exp. Med. 2001193, 123-133. IKK inhibitors of the invention are tested for their ability to block the IgE- or LPS-induced TNFα release using commercially available ELISA kits.

Osteoclast Differentiation and Functional Assays

Human osteoclast precursors are obtained as cryopreserved form from Cambrex (Walkersville, Md.). The cells are differentiated in culture based on instructions from the manufacturer. IKK inhibitors of the invention are tested for their ability to block the differentiation, bone resorption and collagen degradation as described previously (see Khapli et al., Journal of Immunol. 2003, 171, 142-151; Karsdal et al., J Biol. Chem. 2003, 278, 44975-44987; Takami et al., Journal of Immunol. 2002, 169, 1516-1523).

Rat Models for Rheumatoid Arthritis

Such testing is known in the literature and include a standard rat LPS model as described in Conway et al., “Inhibition of Tumor Necrosis Factor-α (TNF-α) Production and Arthritis in the Rat by GW3333, a Dual Inhibitor of TNF-Converting Enzyme and Matrix Metalloproteinases”, J. Pharmacol. Exp. Ther. 2001, 298(3), 900-908; a rat adjuvant induced arthritis model as described in Pharmacological Methods in the Control of Inflammation (1989) p 363-380 “Rat Adjuvant Arthritis: A Model of Chronic Inflammation” Barry M. Weichman {author of book chapter; Alan R. Liss Inc Publisher}; and a rat collagen induced arthritis model as described in Pharmacological Methods in the Control of Inflammation (1989) p 395-413 “Type II Collagen Induced Arthritis in the Rat” DE Trentham and RA Dynesuis-Trentham {authors of book chapter; Alan R. Liss Inc Publisher}. See also, “Animal Models of Arthritis: Relevance to Human Disease” by Bendele et al., Toxicologic Pathology 1999, 27(1), 134-142.

While we have described a number of embodiments of this invention, it is apparent that our basic examples may be altered to provide other embodiments, which utilize the compounds and methods of this invention. Therefore, it will be appreciated that the scope of this invention is to be defined by the appended claims rather than by the specific embodiments, which have been represented by way of example.

1. A compound of formula (IIa): 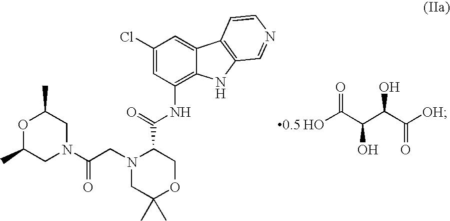
or a solvate thereof.

2. The compound of formula (IIa) according to claim 1, wherein a crystalline form is characterized by at least one of the X-ray powder diffraction peaks at 2θ angles of 3.970°, 5.163°, 6.203°, 7.600°, 7.939°, 14.645°, 19.451° and 22.676°.

3. The compound of formula (IIa) according to claim 1, wherein a crystalline form is characterized by at least one of the following features:
(a-i) at least one of the X-ray powder diffraction peaks shown in Table 1.
(a-ii) an X-ray powder diffraction pattern substantially similar to FIG. 1.
(a-iii) a differential scanning calorimetry (DSC) profile having an endotherm range of about 130° C. to about 160° C.

4. A pharmaceutical composition comprising a pharmaceutically effective amount of a compound according to claim 1, and a pharmaceutically acceptable carrier.

5. A method for treating a patient suffering from, or subject to, an inflammatory disease or immune-related disease comprising administering to said patient a pharmaceutically effective amount of the compound according to claim 1, wherein the inflammatory disease is rheumatoid arthritis, psoriasis, inflammatory bowel disease, chronic obstructive pulmonary disease (COPD) or COPD exacerbations.

6. A compound of formula (IIb): 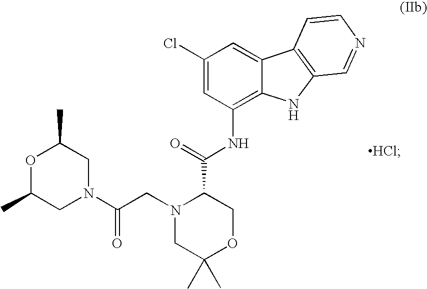
or a solvate thereof.

7. The compound of Formula (IIb) according to claim 6, wherein a crystalline form is characterized by at least one of the X-ray powder diffraction peaks at 2θ angles of 4.809°, 9.687° and 19.440°.

8. The compound of formula (IIb) according to claim 6, wherein a crystalline form is characterized by at least one of the following features:
(b-i) at least one of the X-ray powder diffraction peaks shown in Table 2.
(b-ii) an X-ray powder diffraction pattern substantially similar to FIG. 5.
(b-iii) a differential scanning calorimetry (DSC) profile having an endotherm range of about 160° C. to about 200° C.

9. A pharmaceutical composition comprising a pharmaceutically effective amount of a compound according to claim 6, and a pharmaceutically acceptable carrier.

10. A method for treating a patient suffering from, or subject to, an inflammatory disease or immune-related disease comprising administering to said patient a pharmaceutically effective amount of the compound according to claim 6, wherein the inflammatory disease is rheumatoid arthritis, psoriasis, inflammatory bowel disease, chronic obstructive pulmonary disease (COPD) or COPD exacerbations.

11. A compound of formula (IIc): 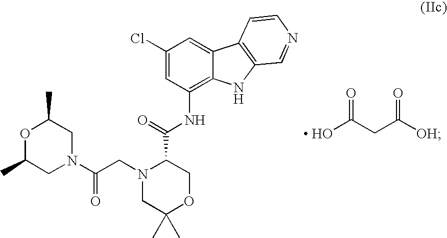
or a solvate thereof.

12. The compound of formula (IIc) according to claim 11, wherein a crystalline form is characterized by at least one of the X-ray powder diffraction peaks at 2θ angles of 4.098°, 16.473° and 20.764°.

13. The compound of formula (IIc) according to claim 11, wherein a crystalline form is characterized by at least one of the following features:
(c-i) at least one of the X-ray powder diffraction peaks shown in Table 3.
(c-ii) an X-ray powder diffraction pattern substantially similar to FIG. 9.
(c-iii) a differential scanning calorimetry (DSC) profile having an endotherm range of about 115° C. to about 170° C.

14. A pharmaceutical composition comprising a pharmaceutically effective amount of a compound according to claim 11, and a pharmaceutically acceptable carrier.

15. A method for treating a patient suffering from, or subject to, an inflammatory disease or immune-related disease comprising administering to said patient a pharmaceutically effective amount of the compound according to claim 11, wherein the inflammatory disease is rheumatoid arthritis, psoriasis, inflammatory bowel disease, chronic obstructive pulmonary disease (COPD) or COPD exacerbations.

16. A compound of formula (IId): 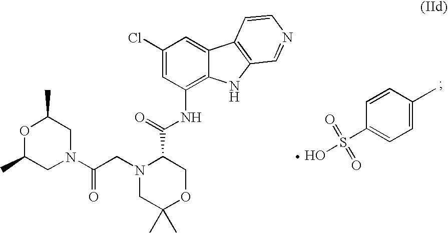
or a solvate thereof.

17. The compound of formula (IId) according to claim 16, wherein a crystalline form is characterized by at least one of the X-ray powder diffraction peaks at 2θ angles of 7.293°, 18.236°, 20.488° and 23.081°

18. The compound of formula (IId) according to claim 16, wherein a crystalline form is characterized by at least one of the following features:
(d-i) at least one of the X-ray powder diffraction peaks shown in Table 4.
(d-ii) an X-ray powder diffraction pattern substantially similar to FIG. 12.
(d-iii) a differential scanning calorimetry (DSC) profile having an endotherm range of about 25° C. to about 105° C.
(d-iv) a differential scanning calorimetry (DSC) profile having a second endotherm range of about 130° C. to about 165° C.

19. A pharmaceutical composition comprising a pharmaceutically effective amount of a compound according to claim 16, and a pharmaceutically acceptable carrier.

20. A method for treating a patient suffering from, or subject to, an inflammatory disease or immune-related disease comprising administering to said patient a pharmaceutically effective amount of the compound according to claim 16, wherein the inflammatory disease is rheumatoid arthritis, psoriasis, inflammatory bowel disease, chronic obstructive pulmonary disease (COPD) or COPD exacerbations.
